(19) United States
(12) Patent Application Publication
(10) Pub. No.: US 2026/0026946 A1
(43) Pub. Date: Jan. 29, 2026

(54) Modular Acetabular Cup System

(71) Applicant: RR Medical Pty Ltd, Toorak VIC (AU)

(72) Inventor: Rodney Ian Walter Richardson, Toorak (AU)

(73) Assignee: RR Medical Pty Ltd, Toorak VIC (AU)

(21) Appl. No.: 18/932,993

(22) Filed: Oct. 31, 2024

(30) Foreign Application Priority Data
Jul. 26, 2024 (AU) 2024205142

(51) Int. Cl. A61F 2/46; A61F 2/30; A61F 2/34

(52) U.S. Cl. CPC A61F 2/4609; A61F 2002/30332; A61F 2002/30405; A61F 2002/30495; A61F 2002/3085; A61F 2002/30878; A61F 2002/3414; A61F 2002/4627

(57) ABSTRACT

A prosthetic acetabular liner including a wall that extends from an apex portion to a circumferential rim portion, the wall having: a convex outer surface; a threaded spigot extending outwardly from the apex portion; a concave inner bearing surface; and an opening surrounded by the circumferential rim portion. The liner is rotatably insertable into an acetabular outer shell having a corresponding threaded apical hole, and rotation of the liner in the shell causes the threaded spigot to engage the threaded apical hole and thereby draw the liner into the shell.

This application claims under 35 U.S.C. § 119(a) the benefit of the filing date of Australian Patent Application No. 2024205142, filed on Jul. 26, 2024, the entire contents of each of which are incorporated herein by reference.

### BACKGROUND OF INVENTION

### Technical Field

This invention relates to a prosthetic hip implant system with a modular acetabular cup. The invention also relates to an acetabular liner component for use in such a system, and an instrument for inserting an acetabular liner into an outer shell for such a system.

### Background Art

Hip replacement surgery is considered a primary treatment option for individuals suffering from conditions such as osteoarthritis, rheumatoid arthritis, fractures, or other forms of hip degeneration. Prosthetic hip implants are often used in such scenarios to replace damaged or diseased hip joints, restoring mobility, alleviating pain, and improving the quality of life for affected patients.

In hip replacement surgery, damaged joint surfaces are replaced with a range of components in order to restore function. These components can be split into two groups: the acetabular component and the femoral component.

The femoral component generally includes a stem portion, a neck portion permanently coupled to or integrally formed with the stem portion, and a part-spherical head coupled to the neck portion. To implant the stem portion, the head of the femur is resected, exposing the intramedullary space of the femur. The stem portion of the femoral component may then be inserted within the intramedullary space. This portion provides a stable fixture for the prosthetic femoral head to attach via a neck portion. The femoral head is designed to be attached to the neck portion of the femoral component, and to act as the articulating surface within the acetabular component.

The acetabular component is designed to replace the damaged socket (acetabulum) of the hip joint. It serves as the cup-shaped portion that fits into the pelvis to create a new functioning hip joint. The acetabular component plays several essential roles in ensuring the success and longevity of the hip replacement.

Firstly, it provides a smooth and low-friction surface that allows for movement of the femoral head component within the socket, mimicking the natural hip joint's range of motion. This reduces friction and wear, which are crucial factors in the implant's durability over time.

Secondly, the acetabular component must be securely fixed to the pelvis to withstand the forces exerted during daily activities like walking, standing, and bending. Various fixing methods, including press-fit, cemented, or hybrid techniques, are employed based on the patient's bone quality and the specific design of the implant.

Lastly, modern acetabular components often incorporate features to promote bone ingrowth or osseointegration, enhancing long-term stability and reducing the risk of implant loosening or dislocation. These advancements underscore the importance of the acetabular component in restoring mobility, alleviating pain, and improving the overall quality of life for patients undergoing hip replacement surgery.

The acetabular component may be one part that is both fixed to the acetabulum at its external surface, as well as providing a smooth bearing surface for the femoral head on its inner surface. This design may be referred to as a monoblock component. Alternatively, the acetabular component may be modular and include a plurality of parts. Commonly, the acetabular component may include an acetabular shell and a liner. The acetabular shell is the component that is fixed to a patient's acetabulum, whilst the liner is what provides a smooth bearing surface configured to accept a femoral head. The acetabular shell is generally a metal material, and the liner may be a metal, polyethylene, or ceramic. This modular arrangement of components allows for a greater degree of flexibility and custom matching of component sizes suitable to a patient's anatomy and surgeon's goals.

In addition to the aforementioned components, another bearing component may house the femoral head, and articulate within the inner surface of the acetabular component. The bearing surfaces in this scenario may include an inner bearing surface of the acetabular component against an outer bearing surface of the bearing component, as well as an inner surface of the bearing element against an outer surface of the femoral head. The bearing component that houses the femoral head may be referred to as a bipolar head, constrained liner, inner liner, or a dual mobility component.

There are several modes of failure that can occur in hip implants, with many relating to dislocation or loosening of the prosthetic components. Such failures can include the femoral head, with or without an additional bearing component, dislocating from the acetabular component. The acetabular shell can also be the subject of failure, with the potential for it to loosen from osteolysis of the native acetabulum, or fail due to component wear, or fracture from trauma. A further mode of dislocation may occur by separation of the shell and liner of a modular acetabular component. Metal liners may disengage, and ceramic liners may fracture. Polyethylene liners rarely fail in this way, but have the potential of failure by, for example, polyethylene wear or deformation.

Each of these modes of failure may be addressed by a range of modifications. For example, to avoid dislocation of the shell from the acetabulum, a range of fixing screws may be used, or the shell may be cemented in place. Both options are a standard practice.

With regard to preventing dislocation of the femoral head, there are a range of strategies a surgeon may use. A common approach to this is choosing the largest possible femoral head size. This increases the jump distance, being the center translation distance required for a femoral head to dislocate from an acetabular component. A consequence of this is that the bearing surface, often a liner, of the acetabular component is often made as thin as possible in order to maximize the size of the femoral head that may be inserted. This can have impacts on the wear of the bearing surface (liner), and may lead to failure of this component.

As shown in U.S. Pat. Nos. 8,398,718 and 8,465,549, the entire disclosure of each of which are incorporated herein by reference, dislocation of a femoral head may be addressed through the use of a constrained liner, particularly one with an inbuilt locking ring. The constrained liner allows for a wide range of motion whilst limiting the chance of dislocation through angled ramps and flanges securing the liner and femoral head at its terminal range within the acetabular component. Further, the inbuilt locking ring mechanism reduces the risk of the femoral head dislocating from the constrained liner.

These methods of failure, being those of an acetabular shell loosening, and the femoral head, with or without a housing bearing element, dislocating, are well-addressed in the literature. The dislocation of an acetabular liner from an acetabular shell, however, is not.

Current methods of engaging a liner into an acetabular shell include impaction following visual and tactile alignment of the rims of each component. Some liners, particularly those made of polyethylene, will have radial protrusions from the rim portion to facilitate proper alignment. For liners of other materials, such as metal or ceramic, alignment is dependent on the surgeon's analysis and placement during the surgery. Once the surgeon is satisfied with the alignment, an instrument is used to apply force to the liner to impact the liner into the shell. If the alignment of the shell and liner are incorrect for a ceramic liner, the liner is likely to shatter, alerting the surgeon to the fact that the components were not properly placed. For a metal liner, however, it is the case that the liner may appear during surgery to be engaged and correctly seated in the shell, however, under physiological load, dislocation can easily occur should this alignment be incorrect.

The proper alignment of an acetabular shell and liner is of critical importance, as their engagement depends on this alignment. In some systems, the shell and liner are engaged via a Morse taper, wherein a tapered section of the outer surface of the liner abuts a correspondingly tapered section of the inner surface of the shell, with the tapered surfaces mating to facilitate this engagement.

Should the alignment of the acetabular shell and liner be incorrect, the Morse taper will not engage as intended. When these components are placed under a physiological load, there is a high chance of failure wherein the liner disengages from the shell.

Presently there are a number of ways in which the alignment of a liner and a shell may be achieved. For example, protrusions provided on the liner may be configured to engage with corresponding indentations on the shell prior to impaction. Further a component known as a locking ring may be used, configured to engage with the shell and liner to ensure alignment.

These alignment methods, however, have no function in maintaining the engagement between the acetabular shell and liner. It would be desirable to provide an alternative and/or better alignment mechanism that also assists to maintain engagement between the shell and liner, especially where a Morse taper provides said engagement.

### SUMMARY OF THE INVENTION

In broad terms, the invention provides an acetabular liner configured to be attached to an acetabular shell via a screw mechanism, improving engagement of the Morse taper between the two components.

In a first aspect, the invention provides a prosthetic acetabular liner including a wall that extends from an apex portion to a circumferential rim portion, the wall having: a convex outer surface; a threaded spigot extending outwardly from the apex portion; a concave inner bearing surface; and an opening surrounded by the circumferential rim portion; wherein the liner is rotatably insertable into an acetabular outer shell having a corresponding threaded apical hole, and rotation of the liner in the shell causes the threaded spigot to engage the threaded apical hole and thereby draw the liner into the shell.

This provides the advantage of aligning the acetabular liner with the acetabular shell. As the liner is rotated within the shell, the engagement of the threaded spigot within the threaded apical hole draws the liner into the shell.

In some embodiments, when the liner is rotated and drawn into the shell, the outer surface of the liner to abuts a portion of an inner surface of the acetabular outer shell.

In a further embodiment, the liner and acetabular shell are secured by a Morse taper, including a tapered section on the outer surface of the liner acting upon a corresponding tapered section on the inner surface of the acetabular outer shell.

In yet a further embodiment, the thread of the spigot within the threaded apical hole creates a compressive force on the Morse taper.

In addition to ensuring correct alignment of the shell and liner, the threaded spigot exerting a compressive force on the Morse taper further engages the liner and shell. This has the benefit of reducing the likelihood of failure of the acetabular component that may occur due to loss of engagement between the liner and shell.

In a further aspect, the invention provides an instrument for inserting an acetabular liner into an outer shell of a prosthetic hip implant system, the liner having a circumferential rim portion with a plurality of indentations to enable gripping and the liner being rotatably insertable into the outer shell, the instrument including: a drive head configured to apply a torque to the circumferential rim portion of the liner by means of a plurality of protrusions extending from a peripheral edge of the drive head, the protrusions being arranged to engage corresponding indentations of the liner; and a drive shaft to enable a user to rotate the drive head about a central axis and apply a desired torque to the drive head.

In some embodiments, the instrument may have a drive head including an inner drive element for applying the torque to the circumferential rim portion of the liner, the protrusions being provided on the inner drive element; and a coaxially outer counter-drive element for applying a counter-torque to a circumferential rim portion of the outer shell, the counter-drive element having a plurality of projections for engaging corresponding notch portions provided in the circumferential rim portion of the outer shell, and wherein the drive shaft includes: an inner shaft connected to the inner drive element; and a hollow outer shaft, coaxially surrounding the inner shaft, and connected to the counter-drive element.

In a still further aspect, the invention provides a prosthetic hip implant system including: a prosthetic femoral component including a stem portion, a neck portion coupled to the stem portion and a part-spherical head coupled to the neck portion; and an acetabular component including: an outer shell having: a shell wall that extends from an apex portion to a circumferential rim portion; a convex outer surface configured to engage the acetabulum; a concave inner surface; an open end surrounded by the circumferential rim portion; and a threaded apical hole at the apex portion which extends from the inner surface toward the outer surface of the outer shell; a liner having: a wall that extends from an apex portion to a circumferential rim portion; a convex outer surface; a threaded spigot extending outwardly from the apex portion; a concave inner bearing surface; and an open end surrounded by the circumferential rim portion; and a bearing element having: a part-spherical outer bearing surface configured to engage the inner bearing surface of the liner; and a part spherical inner surface having an open end for receiving the head of the femoral component wherein the liner is rotatably insertable into the outer shell such that rotation of the liner causes the threaded spigot to engage the threaded apical hole and thereby draw the liner into the outer shell.

Those skilled in the art will appreciate that the present disclosure allows for a modular acetabular component having a reduced risk of dislocation between an outer shell and a liner, with a tool further provided for the facilitation of the same. Further, when combined in a system with a bearing element further described in the following description of embodiments, the chance of multiple modes of failure is reduced.

Various features, aspects, and advantages of the invention will become more apparent from the following description of embodiments of the invention, along with the accompanying drawings in which like numerals represent like components.

### BRIEF DESCRIPTION OF THE DRAWINGS

Embodiments of the invention will now be described with particular reference to the accompanying drawings. However, it is to be understood that the features in and described with reference to the accompanying drawings are illustrated by way of example, and not by way of limitation, of which:

FIG. 1 is an exploded view of an embodiment of an acetabular component of a hip replacement with an outer shell, a liner, and a bearing element;

FIG. 2 shows a cross-sectional view of the liner of FIG. 1 with a protruding threaded spigot forming an apical screw;

FIG. 3 is a cross-sectional view of the outer shell of FIG. 1 having a threaded apical hole;

FIG. 4 is a cross-sectional view, illustrating the assembly of the acetabular component of FIG. 1 including the outer shell, liner, and bearing element, wherein the apical screw of the liner is fully engaged with the apical hole of the shell;

FIG. 5 is a bottom view of the liner of FIG. 2 mounted within the outer shell of FIG. 3 to form an acetabular cup of FIG. 1 to which a bearing element may be mounted within;

FIG. 6 is a side view of a portion of an embodiment of an instrument configured to engage with the liner of FIG. 2 or FIG. 5;

FIG. 7 is a partial cross-sectional view of the acetabular cup of FIG. 5 and the instrument of FIG. 6 configured to engage with the liner of FIG. 2;

FIG. 8 shows a detailed view of a second embodiment of an instrument configured to engage with each of the liner of FIG. 2 and the outer shell of FIG. 3;

FIG. 9 is a view of the instrument of FIG. 8, showing engagement with other surgical instruments; and

FIG. 10 shows a cross-sectional view of a system including a second embodiment of a liner, a second embodiment of a bearing element, and an embodiment of a part femoral component.

The invention will now be described more fully hereinafter with reference to the accompanying drawings, in which various embodiments, although not the only possible embodiments, of the invention are shown. The invention may be embodied in many different forms and should not be construed as being limited to the embodiments described below.

### DETAILED DESCRIPTION OF EMBODIMENTS

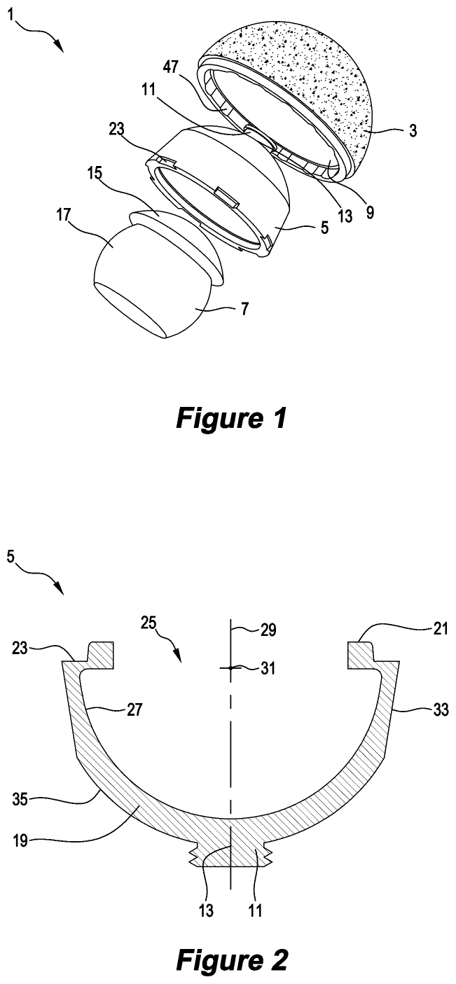

Referring to FIG. 1, there is shown an exploded view of a three-part acetabular component or shell generally denoted as 1 with an outer shell 3, a liner 5, and a bearing element 7. Outer shell 3 is shown having a shell inner surface 9 configured to accept the liner 5. The liner 5 is shown having a threaded spigot 11 at a liner apex portion 13 forming an apical screw. Bearing element 7 is shown as having two part-spherical surface regions 15, 17.

In this embodiment, the acetabular shell 1 is preferably made of a metal or metal alloy. However, it may be made of materials other than a metal or metal alloy. Further, the shell 1 may have features not shown in FIG. 1 that provide a mechanism for fixing to an acetabulum, such as holes configured to take screws.

The bearing element 7 may be made of polyethylene, ceramic, or any other material suitable for a bearing surface in a hip replacement. It may have a singular part-spherical outer surface rather than the two part-spherical outer surfaces shown in FIG. 1.

FIG. 2 shows a cross-sectional view of the liner 5 of FIG. 1. The liner 5 includes a liner wall 19 that extends from the liner apex portion 13 to a liner rim portion 21. At the liner apex portion 13 is a threaded spigot 11 projecting outwardly to form an apical screw. In this embodiment, the liner rim portion 21 has a plurality of indentations 23 which may engage with a surgical instrument. The area surrounded by the liner rim portion 21 provides a liner opening 25 wherein the bearing element 7 may be inserted. The bearing element 7 may interact with a liner inner bearing surface 27. The liner 5 has a liner central axis 29 defined as the axis extending from a center point 31 of the liner opening 25 to the liner apex portion 13. In this embodiment there is also provided an outer surface tapered section 33 on a liner outer surface 35 configured to engage with the shell inner surface 9.

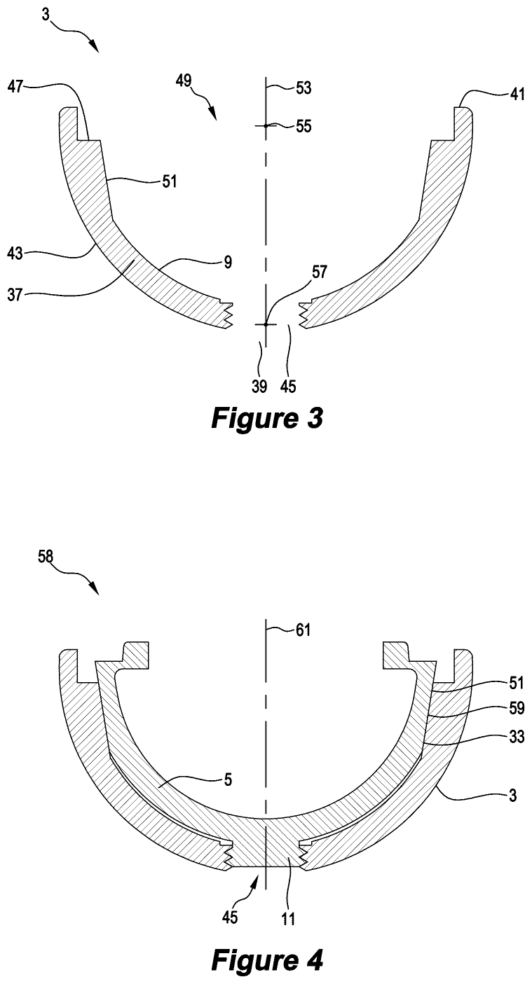

Referring to FIG. 3 there is shown a cross-sectional view of the outer shell 3 of FIG. 1. The outer shell 3 includes a shell wall 37 that extends from a shell apex portion 39 to a circumferential shell rim portion 41. A shell outer surface 43 provides a convex surface for fixing to an acetabulum. At the shell apex portion 39 is a threaded apical hole 45. In this embodiment, the shell rim portion 41 has a plurality of notches 47 which may engage with a surgical instrument. The area surrounded by the shell rim portion 41 provides a shell opening 49 wherein a liner 5 may be inserted. The liner outer surface 35 (FIG. 2) may interact with the shell inner surface 9. In this embodiment there is also provided an inner surface tapered section 51 on the shell inner surface 9 configured to engage with the liner outer surface 35, and in particular with the outer surface tapered section 33 (FIG. 2) on the liner outer surface 35. A shell central axis 53 is defined as the axis extending from a center point 55 of the shell opening 49 to a center point 57 of the shell apex portion 39.

The threaded apical hole 45 in this embodiment may be used for purposes such as receiving an instrument to hold the shell in a stationary position whilst fixing the shell to the acetabulum via screws, cement, or other methods. Those skilled in the art will appreciate that the presence of an apical hole on an acetabular shell is common for this purpose. The apical hole 45 is shown in this embodiment as extending through the shell wall from the inner surface to the outer surface, however the hole does not need to extend the entire way to the outer surface.

Turning to FIG. 4, there is shown a cross-sectional view of an assembled acetabular cup generally denoted as 58 including the outer shell 3 of FIG. 3 and the liner 5 of FIG. 2. Shown in this view is how the outer shell 3 and liner 5 are configured to engage with one another. Specifically, it shows how the apical screw of the threaded spigot 11 engages with the threaded apical hole 45, with these two features being complementary to one another. When the threaded spigot 11 is fully engaged in the threaded apical hole 45 the liner 5 can be considered as fully seated in the outer shell 3. In this state, the liner 5 is drawn into outer shell 3 and a Morse taper 59 engages. The Morse taper 59 includes the outer surface tapered section 33 and the inner surface tapered section 51. The Morse taper 59 provides a method of securing the liner 5 and outer shell 3 together. The thread of the spigot 11 within the threaded apical hole 45 creates a compressive force on the Morse taper, strengthening the engagement of the liner 5 and outer shell 3.

Though liners in general may be made of materials such as polyethylene and ceramic, presently these are not suitable for use as a screw engaging with a metallic threaded hole such as that of an acetabular shell. As such, the liner of the invention should predominantly include a metal or metal alloy. This should not, however, discount the potential for elements such as ceramic coating to be included in some embodiments.

Further to the above, the view in FIG. 4 displays how the central axes 29 and 53 of the liner 5 and outer shell 3, respectively, are coaxial, forming a common axis 61 when these two parts are assembled. Rotation about the common axis 61 is what allows the threaded spigot 11 to engage with the threaded apical hole 45.

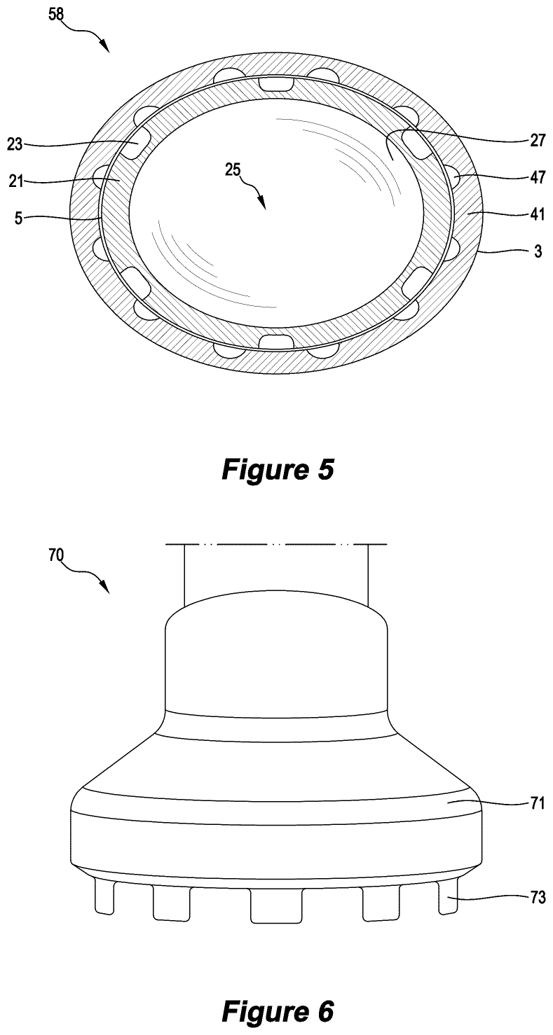

Referring to FIG. 5, the assembled acetabular cup 58 is viewed from the liner opening 25. The seating of liner 5 into outer shell 3, the rim portions 21 and 41 of the liner 5 and outer shell 3, respectively, are highlighted. In this embodiment, the liner rim portion 21 contains a plurality of indentations 23, and the shell rim portion 41 contains a plurality of notches 47. Many outer shells have a rim portion with notches to facilitate alignment of polyethylene or other liners. In this embodiment, the notches 47 may be used to engage a surgical instrument. The plurality of indentations 23 provided on the liner 5 may similarly engage a surgical instrument. Further, such an instrument may be used to apply torque to at least one of the liner 5 or the outer shell 3, twisting them about the common axis 61.

In this embodiment, the liner rim portion 21 contains a plurality of indentations 23 placed on the periphery (i.e. the radially outer edge) of the rim portion. These indentations may be placed anywhere on the rim portion 21 accessible by a surgical instrument. Similarly, the notches 47 on the shell rim portion 41 are placed radially inward, and may too be placed anywhere on the shell rim portion 41 that may be accessible by a surgical instrument.

Those skilled in the art will appreciate that liners detailed in the prior art do not generally have a liner rim portion with geometry capable of having such indentations 23, much less having indentations capable of engagement with an instrument used to apply torque for twisting the liner into an acetabular outer shell. An embodiment of a liner with a rim portion capable of having such indentations will be further detailed with reference to FIG. 10.

FIG. 6 shows an embodiment of an instrument 70 with a drive head 71 including a plurality of peripheral protrusions 73 configured to engage with the liner indentations 23.

In this embodiment the peripheral protrusions 73 extend axially. In other embodiments they may extend radially inwardly or outwardly, according to the specific position and orientation of indentations in a corresponding liner. The number of protrusions may vary. The shape of the instrument may also vary.

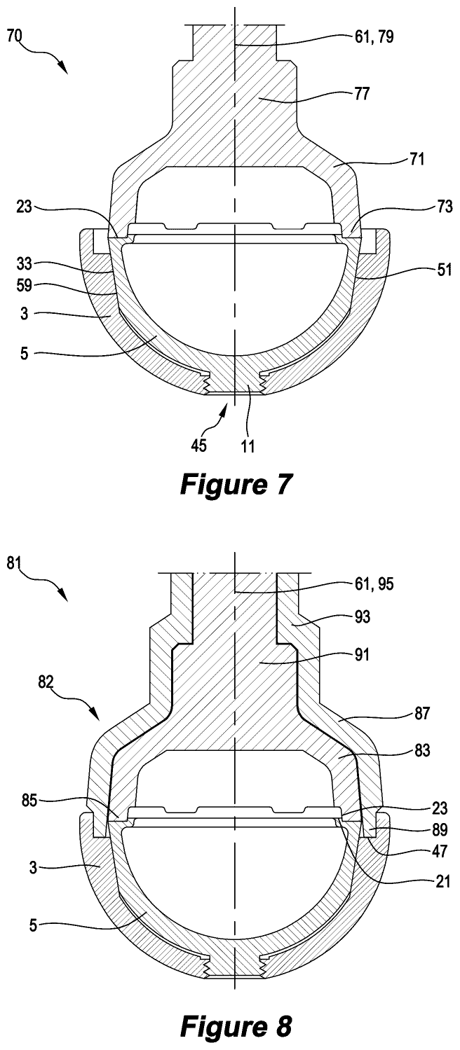

FIG. 7 provides further details of the embodiment of the instrument 70 of FIG. 6 for inserting an acetabular liner 5 into an acetabular shell 3, configured to engage a plurality of peripheral protrusions 73 with liner indentations 23. Note that FIG. 7 shows an embodiment of a bearing element while a cross-sectional view of the instrument 70, outer shell 3, and liner 5 are shown. This embodiment includes a drive shaft 77 to enable a user to rotate the drive head 71 about a central axis 79, which is coaxial with the common axis 61, and apply a desired torque to the acetabular liner 5 by means of the drive head 71. By applying torque to the liner 5 using instrument 70, the threaded spigot 11 engages with the threaded apical hole 45. Once engaged, the liner 5 is drawn into the outer shell 5, engaging the Morse taper 59 between outer surface tapered section 33 and the inner surface tapered section 51. This creates a compressive force on the Morse taper 59, strengthening the engagement of the liner 5 and outer shell 3

FIG. 8 shows an embodiment of an alternative instrument 81 for inserting an acetabular liner 5 into an acetabular outer shell 3 where a drive head 82 includes an inner drive element 83 configured to engage liner indentations 23 and thus apply the torque to the liner rim portion 21, with a plurality of peripheral protrusions 85 being provided on the inner drive element 83. The drive head 82 also includes a coaxially outer counter-drive element 87 for applying a counter-torque to the shell rim portion 41 with the counter-drive element 87 having a plurality of projections 89 for engaging corresponding shell notches 47. Note that FIG. 8 shows an embodiment of a bearing element while a cross-sectional view of the instrument 81, acetabular outer shell 3, and liner 5 are shown.

In this embodiment, the drive shaft 90 includes an inner shaft 91 connected to the inner drive element 83 and a hollow outer shaft 93, coaxially surrounding the inner shaft 91, and connected to the counter-drive element 87. The rotation of the shafts occurs around a central axis 95, which is coaxial with the common axis 61.

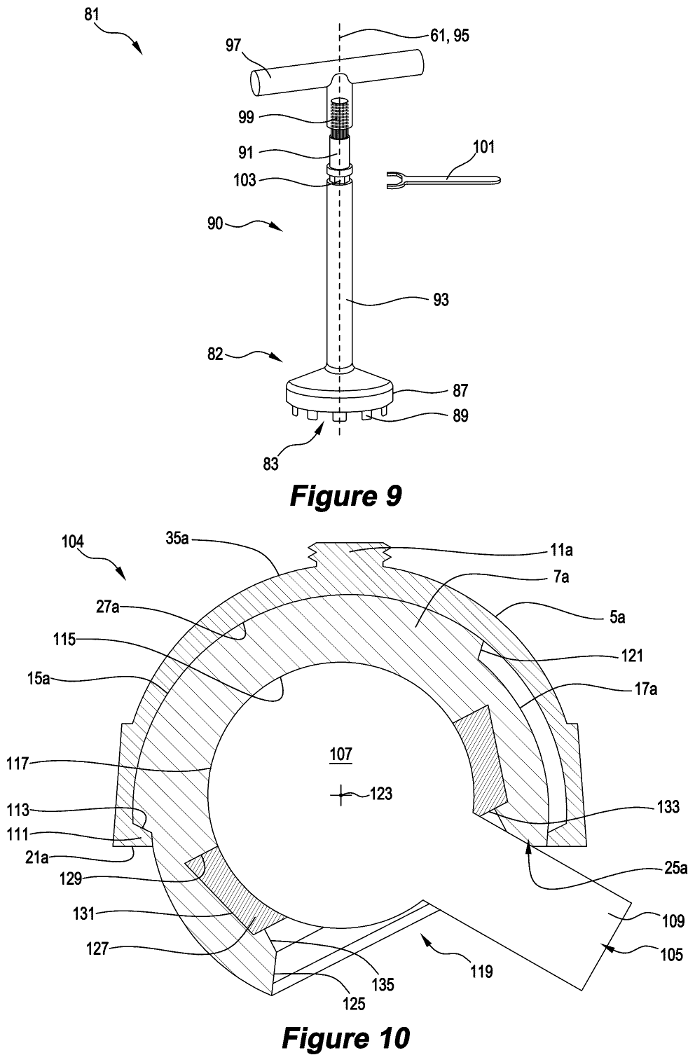

Turning to FIG. 9, it is shown how further instruments may be attached to instrument 81 to apply a force to the inner shaft 91 and the hollow outer shaft 93. A torque limiter 97 may attach to an engagement area 99 of the inner shaft 91. This torque limiter 97 may be a standard instrument and may be used to apply a predetermined force to the inner shaft to enable the screwing action of the threaded spigot 11a within a threaded apical hole. A removable or permanent handle 101 may also engage with the hollow outer shaft 93 by an engagement area 103. Such a handle 101 may be a simple protrusion manufactured as part of the instrument 81, or it may be a wrench or similar tool that provides a means of gripping the hollow outer shaft or a specific an engagement area 103 of the same. Note that torque limiter 97 may attach to an engagement area 99 of the inner shaft 91, wherein inner shaft 91 is connected to the inner drive element 83. Inner drive element 83 is configured to engage liner indentations 23 and, thus, apply the torque to the liner rim portion 21, with a plurality of peripheral protrusions 85 being provided on the inner drive element 83. Handle 101 may engage with the hollow outer shaft 93 by an engagement area 103. The hollow outer shaft 93 is connected to a counter-drive element 87 that has a plurality of projections 89 for engaging corresponding shell notches 47.

When using the instrument 81, it may be desirable to use the counter-drive element 87 and its associated hollow outer shaft 93 and handle 101 to hold the acetabular shell 3 in a stationary position so as to not disturb any fixing mechanism such as screws or cement that have been applied between the shell 3 and a patient's acetabulum.

With reference to FIG. 10, there is an embodiment of a system 104 including a liner 5a, a bearing element 7a, and a part femoral component 105. This system may include further prosthetic components such as an acetabular shell and a femoral stem. The liner outer surface 35a may engage with an acetabular shell having an apical hole, wherein a threaded spigot 11a is configured to engage with the apical hole. The part femoral component 105 comprises a femoral head 107 and a femoral neck 109, where the femoral neck may join to a femoral stem.

In this embodiment the liner 5a has a liner rim portion 21a which includes a flange 111 extending radially inwardly. The flange 111 has a circumferential bearing-facing surface 113. The flange may vary in size and shape, and angle at which it extends radially inwardly.

Bearing element 7a is shown mounted within a liner opening 25a. Bearing element 7a has a first part-spherical bearing surface region 15a engaging the liner inner bearing surface 27a. Bearing element 7a includes a part-spherical inner bearing surface 115 designed to receive an outer bearing surface 117 of the femoral head 107 through an opening 119. As is typical, the femoral head 107 engages with and may rotate in any direction on inner bearing surface 115 within bearing element 7a.

The first part-spherical surface region 15a is capable of rotating on the liner inner bearing surface 27a in any direction and in FIG. 10 is shown rotated counter clockwise within liner 5a as a result of movement of the part femoral component 105 having the femoral head 107 mounted in the bearing element 7a. Bearing element 7a further includes a second part-spherical surface region 17a which extends from the first part-spherical bearing surface region 15a but has a smaller diameter and is spaced radially inwardly from liner inner bearing surface 27a.

In a preferred embodiment the part-spherical bearing surface regions 15a and 17a are concentric and having the same center, for example bearing center 123 thus forming a stepped contact surface 121 between part-spherical bearing surface regions 15a and 17a.

During use, bearing element 7a rotates within liner 5a on the liner inner bearing surface 27a and the femoral head and neck 107 and 109 rotate within the bearing element 7a on inner bearing surface 115. When the femoral neck 109 makes contact with bearing element 7a it engages a rim portion 125 which extends around opening 119 of the bearing element 7a. When the femoral neck 109 engages rim portion 125 the bearing element 7a is rotated within liner 5a, which rotation is limited by the engagement of stepped contact surface 121 and bearing-facing surface 113 of flange 111. This arrangement may be referred to as a constrained system.

Further, the flange 111 may act to provide a thicker liner rim portion 21a, thereby creating a geometry with the means of having a plurality of notches (not shown) for engagement with the protrusions 73 or 85 of a drive head 71 or 83 of a surgical instrument 70 or 81 (FIGS. 6-8). This arrangement, as previously described, may allow the liner 5a to be rotatably inserted into an acetabular shell.

In a further embodiment, the inner bearing surface 115 of bearing element 7a includes an outwardly extending recess 127 having upper end wall 129 extending laterally outwardly to an outer sloping wall 131 of recess 127 which slopes downwardly and inwardly towards opening 119 through which the femoral head 107 is inserted. Outer sloping wall 131 terminates in a lower end wall 133 which extends laterally inwardly and abuts a peripheral edge wall 135 forming part of opening 119 in the bearing element 7a. Peripheral edge wall 135 is spaced inwardly of opening 119 and is connected to part-spherical surface region 17a by rim portion 125.

As shown in U.S. Pat. No. 4,798,610, the entire disclosure of which is incorporated herein by reference, a further embodiment may include a split ring (not shown), preferably constructed of polyethylene similar to that used to construct the bearing element 7a, has a split portion, rendering the ring radially expandable and contractable. The ring may include an upper end wall, a downwardly and inwardly sloping outer wall of the same slope as outer sloping wall 131 of recess 127 for mating engagement therewith.

At assembly, the femoral head 107 is inserted axially into opening 119 in bearing element 7a, past peripheral edge wall 135 and, where included, through a split ring, so that ring will be raised axially upwardly to an upper position within recess 127, expanding the ring. Once the equator or largest diameter of the femoral head 107 presses through the ring into engagement with inner bearing surface 115 of bearing element 7a, the ring may contract. When the femoral head 107 contacts inner bearing surface 115, the ring contracts and slides downwardly along outer sloping wall 131, capturing the femoral head 107 in bearing element 7a. Other methods of retaining the ball within the bearing element may be used such as that shown in U.S. Pat. No. 7,455,694 the entire disclosure of which is incorporated herein by reference.

The femoral head 107 is coupled to the femoral neck 109 either as a one-piece construction or via any suitable rigid mechanical connection such as a Morse taper. The neck may be integrally formed with, or connected by any suitable rigid connection, such as a Morse taper, to an intramedullary stem. Such modular femoral component designs are well known. The stem is generally intended to be received within a medullary canal of the femur although in certain oncological applications may form the entire proximal femur.

Without further elaboration, it is believed that one skilled in the art can use the preceding description to utilise the claimed inventions to their fullest extent. The examples and embodiments disclosed herein are to be construed as merely illustrative and not a limitation of the scope of the present disclosure in any way.

It will be apparent to those having skill in the art that changes may be made to the details of the above-described embodiments without departing from the underlying principles discussed. As such, various modifications and improvements of the embodiments specifically disclosed in the description above are within the scope of the appended claims. For example, any suitable combination of features of the various embodiments described is contemplated.

As used herein and in the appended claims, the singular form of a word includes the plural, unless the context clearly dictates otherwise. Thus, the references “a,” “an” and “the” are generally inclusive of the plurals of the respective terms. For example, reference to “a feature” includes a plurality of such “features.” The term “and/or” used in the context of “X and/or Y” should be interpreted as “X,” or “Y,” or “X and Y”.

It is to be understood that, if any prior art publication is referred to herein, such reference does not constitute an admission that the publication forms a part of the common general knowledge in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A prosthetic acetabular liner comprising:
a wall that extends from an apex portion to a circumferential rim portion, the wall comprising:
a convex outer surface;
a threaded spigot extending outwardly from the apex portion;
a concave inner bearing surface; and
an opening surrounded by the circumferential rim portion;
wherein the prosthetic acetabular liner is rotatably insertable into an acetabular outer shell having a corresponding threaded apical hole, and rotation of the prosthetic acetabular liner in the acetabular outer shell causes the threaded spigot to engage the threaded apical hole and thereby draw the prosthetic acetabular liner into the acetabular outer shell.

2. The prosthetic acetabular liner of claim 1, wherein the convex outer surface of the prosthetic acetabular liner is configured to abut a portion of an inner surface of the acetabular outer shell.

3. The prosthetic acetabular liner of claim 2, wherein the prosthetic acetabular liner and the acetabular outer shell are secured by a Morse taper, comprising a tapered section on the outer surface of the prosthetic acetabular liner acting upon a corresponding tapered section on the inner surface of the acetabular outer shell.

4. The prosthetic acetabular liner of claim 3, wherein a thread of the threaded spigot within the threaded apical hole creates a compressive force on the Morse taper.

5. The prosthetic acetabular liner of claim 1, wherein a rim portion of the prosthetic acetabular liner has a plurality of indentations configured to be engaged by a surgical instrument.

6. The prosthetic acetabular liner of claim 1, wherein the prosthetic acetabular liner is constructed of a metal or metal composite.

7. The prosthetic acetabular liner of claim 1, wherein a rim portion of the prosthetic acetabular liner comprises a circumferential radially inwardly extending flange.

8. An instrument for inserting an acetabular liner into an outer shell of a prosthetic hip implant system, wherein the acetabular liner comprises a circumferential rim portion with a plurality of indentations to enable gripping and the acetabular liner being rotatably insertable into the outer shell;
the instrument comprising:
a drive head configured to apply a torque to the circumferential rim portion of the acetabular liner by a plurality of protrusions extending from a peripheral edge of the drive head, the plurality of protrusions being arranged to engage corresponding indentations of the acetabular liner; and
a drive shaft that rotates the drive head about a central axis and apply a desired torque to the drive head.

9. The instrument of claim 8, wherein the drive head comprises:
an inner drive element for applying the torque to the circumferential rim portion of the acetabular liner, the plurality of protrusions being provided on the inner drive element; and
a coaxially outer counter-drive element for applying a counter-torque to a circumferential rim portion of the outer shell, the counter-drive element comprising a plurality of projections for engaging corresponding notch portions provided in the circumferential rim portion of the outer shell; and
wherein the drive shaft comprises:
an inner shaft connected to the inner drive element; and
a hollow outer shaft, coaxially surrounding the inner shaft, and connected to the coaxially outer counter-drive element.

10. The instrument of claim 9, wherein an input end of the drive shaft is configured to enable a torque wrench to be attached to the inner shaft to enable application of a torque to the inner drive element.

11. The instrument of claim 10, wherein the torque applied to the inner drive element is predetermined by a torque limiter.

12. The instrument of claim 10, wherein the input end of the drive shaft is configured to enable a handle to be attached to the outer shaft to enable application of a counter-torque to the counter-drive element such that the outer shell can be held in a stationary position as the acetabular liner is inserted into the outer shell.

13. A prosthetic hip implant system comprising:
a prosthetic femoral component comprising a stem portion, a neck portion coupled to the stem portion, and a part-spherical head coupled to the neck portion; and
an acetabular component comprising:
an outer shell comprising:
a shell wall that extends from a first apex portion to a first circumferential rim portion;
a first convex outer surface configured to engage an acetabulum;
a concave inner surface;
a first open end surrounded by the circumferential rim portion; and
a threaded apical hole at the apex portion which extends from the concave inner surface toward the concave outer surface of the outer shell;
a liner comprising:
a liner wall that extends from a second apex portion to a second circumferential rim portion;
a second convex outer surface;
a threaded spigot extending outwardly from the second apex portion;
a concave inner bearing surface; and
a second open end surrounded by the second circumferential rim portion; and
a bearing element having:
a part-spherical outer bearing surface configured to engage the concave inner bearing surface of the liner; and
a part spherical inner surface having an open end for receiving the part-spherical head of the prosthetic femoral component;
wherein the liner is rotatably insertable into the outer shell such that rotation of the liner causes the threaded spigot to engage the threaded apical hole and thereby draw the liner into the outer shell.

14. The prosthetic hip implant system of claim 13, wherein the convex outer surface of the liner is configured to abut a portion of an inner surface of the outer shell.

15. The prosthetic hip implant system of claim 13, wherein the liner and outer shell are secured by a Morse taper, comprising a tapered section on the second convex outer surface of the liner acting upon a corresponding tapered section on the inner surface of the outer shell.

16. The prosthetic hip implant system of claim 15, wherein a thread of the threaded spigot within the threaded apical hole creates a compressive force on the Morse taper.

17. The prosthetic hip implant system of claim 13, wherein the bearing element comprises a first part-spherical outer surface and a second part-spherical outer surface, the first part-spherical outer surface is rotatable on the inner bearing surface of the liner and is defined by a first radius extending from a center of the bearing element, the second part-spherical outer surface being defined by a second radius from the center of the bearing element which has a magnitude that is less than a magnitude of the first radius.

18. The prosthetic hip implant system of claim 13, wherein the bearing element comprises a conically tapered area on the part spherical inner surface.

19. The system of claim 18, wherein the conically tapered area houses a split locking ring.
